(19) United States
(12) Patent Application Publication
(10) Pub. No.: US 2011/0070320 A1
(43) Pub. Date: Mar. 24, 2011

(54) BIODEGRADABLE THERMORESPONSIVE 3-ARM POLYETHYLENE GLYCOL POLY(LACTIDE-CO-GLYCOLIDE) COPOLYMER FOR GINSENG ADMINISTRATION

(76) Inventors: Soonkap Hahn, Irvine, CA (US); Weijian Zhang, Irvine, CA (US); Jae Young Yang, Irvine, CA (US); Younjung Yuk, Irvine, CA (US)

(21) Appl. No.: 12/779,026

(22) Filed: May 12, 2010

Related U.S. Application Data
(63) Continuation-in-part of application No. 12/565,110, filed on Sep. 23, 2009, now abandoned.

(51) Int. Cl. A61K 36/258; A61P 1/04

(52) U.S. Cl. 424/728

(57) ABSTRACT

The present invention discloses a composition comprising a ginseng product, a mucoadhesive and a mucosa penetration enhancer in an aqueous biodegradable block copolymer solution consisting of a 3-arm polyethylene glycol (PEG) and 3 polyesters chains, each of which extends on each of the arm of the 3-arm PEG. The invention also provides a method for transmucosal delivery of pharmaceuticals and nutraceutical such as ginsenosides with improved absorption and bioavailability.

### CROSS-REFERENCES

The present application is a continuation-in-part of patent application Ser. No. 12/565,110 filed on Sep. 23, 2009 entitled “Biodegradable Thermoresponsive 3-Arm Polyethylene Glycol Poly(Lactide-Co-Glycolide) Copolymers” and currently pending. This application is incorporated herein by this reference.

### FIELD OF INVENTION

The present invention relates to water soluble, low molecular weight, thermoresponsive, biodegradable block copolymers and their use for transmucosal delivery of pharmaceuticals and nutraceuticals. Particularly, this invention relates to a composition comprising a block copolymer consisting of a 3-arm polyethylene glycol (PEG) and 3 polyesters chains and its use for transmucosal delivery of ginseng extracts or ginsenosides.

### BACKGROUND OF THE INVENTION

Localized drug delivery and controlled drug release are desirable for many treatments, especially, for anticancer therapy, hormonal therapy, antibiotics therapy, and immunosuppressant therapy. Localized drug delivery decreases toxicity, increases efficacy by maintaining a high drug concentration in targets while minimizing the systemic exposure to toxic drugs. A confined depot system having a controlling mechanism for releasing drug in tissue or a targeted disease site, such as a tumor, is an obvious choice. A depot drug delivery system may also release drugs or biological active agents such as vaccines or antibodies which inflict systemic responses after their absorption into host's circulation system. In general, the depot drug delivery system uses polymeric materials and can be categorized into two groups: non-biodegradable and biodegradable. Non-biodegradable depot systems are stable in physiological conditions, and those in solid forms such as implants need to be removed surgically after completion of drug release. Biodegradable depot systems are degraded in body to nontoxic and absorbable components and therefore need not to be removed. The requirements for an ideal depot system include biodegradability, stability, biocompatibility, a proper drug releasing property, and a suitability for formulation and dose formation. The drug release is modulated by many parameters such as drug and polymer hydrophobicities, polymer degradation rate, molecular weight, crosslinking, erosion mechanism, porosity, size and shape of the polymer matrix. These parameters influence the microstructure of the matrix and consequently the drug diffusion rate in the system.

A solid or semi-solid polymer composition is suitable for diffusion-based delivery system because the movement of the drug molecule is restricted within the solid matrix. However, a surgical implantation is required for a solid drug release depot to be placed in tissue. A solution to this problem would be a liquid polymeric formulation which transforms into a firm depot system in a recipient's body. Many polymeric materials have been considered to meet this criterion. For example, Poly(D,L-lactide or L-lactide) (PLA) and Poly(lactide-co-glycolide) (PLGA) are the most used biodegradable polymers in drug delivery formulations due to their non-toxicity and biodegradability. However, PLA and PLGA are rigid materials and have been fabricated into solid forms such as implants or injectable microspheres as drug delivery systems. The solid implants are surgically implanted in and removed from tissue. Although PLA and PLGA microsphere formulations are injectable they are not suitable for biological drugs. This is because, like most poly(α-hydroxy acid) polyesters, PLA and PLGA are hydrophobic and require organic solvents such as methylene chloride in fabrication which may degrade or denature polypeptide or protein. While other hydrophilic non-toxic organic solvents such as N-methyl pyrrolidone (NMP) have been used in depot systems containing PLA or PLGA, water based drug delivery formulations are preferred for biological drugs because of its non-toxicity, biocompatibility and hydrophilicity. In the hydrophilic environment embedded proteins are less prone to denaturation or loss of activity. Dosages in aqueous gel forms are more biocompatible and less mechanically irritating to the surrounding tissue than other physical forms such as solid polymer implants, films or microspheres. In addition, a firm gel in a tissue serves as a diffusion based drug release depot which has a different release profile as those of liquid dosages or solid implants

The formation of gel structure in water involves different crosslinking mechanisms for different water soluble polymeric materials, including non-covalent interactions such as ion-ion interaction, protein/peptide coil formation, and micelle formation, and covalent bond formation between polymer chains. A non-covalent crosslinking is preferred over the chemical crosslinking due to potential material toxicity or polymer drug reactions. Water soluble polymers are either hydrophilic polymers such as PEG and its related derivatives or amphiphilic polymers consisting of hydrophilic polymer blocks and hydrophobic polymer blocks. PEG is readily soluble in water via the solvation of its ethylene glycol units while amphiphilic block copolymers form aqueous solutions through the formation of more stable micelles, where the hydrophobic polymer chains form the cores and the hydrophilic chains face the aqueous phase. An amphiphilic block copolymer has the benefit of being compatible with biological drugs such as proteins and peptides and also has the ability to increases the solubility of many insoluble or less soluble small molecules. PEG-polyester block polymers have been shown to increase solubility of paclitaxel and other anticancer drugs.

A thermoresponsive polymer has the property of changing the form of its aqueous solution responding to temperature change. An aqueous solution of a polymer with a sol-gel transition property exists as an aqueous solution below its gel transition temperature (also known as lower critical solution temperature, LCST) and solidifies as a gel when the temperature reaches its LCST. This reverse gelation property has been explored by using a polymeric formulation which is prepared as an aqueous solution of a thermoresponsive polymer containing a drug and injected through a needle to a location in the recipient's body and transformed to a gelled semi-solid form at the body temperature (e.g. 37° C. for human beings). The gel will remain as a semi-solid for a period of time from several hours to weeks during which the drug is released from the depot system. The integrity and properties of the polymer matrix change as the polymer degrades. After the polymer is significantly degraded, it becomes a liquid and eventually is absorbed by the surrounding tissue.

A biodegradable thermoresponsive synthetic block copolymer is usually amphiphilic and contains hydrophilic and hydrophobic components. This type of amphiphilic copolymer can be dissolved in cold water and when gradually heated and the temperature reaches their LCST, the solution changes phase from solution to gel with a dramatic increase in viscosity. The gelling mechanism has been postulated that at low temperature the amphiphilic polymer chains in aqueous solution form micelles with the hydrophobic ends aggregated as the cores and the hydrophilic ends facing to the polar phase. At this stage the hydrophilic polymer chains are fully hydrated through hydrogen bonding with water molecules and the micelles are completely dissolved. When temperature rises, the water molecules in the hydrogen bonding escape and the hydrophobic interaction between polymer chains becomes significant which causes the micelles to be “bridged” or physically crosslinked. At this stage the solution changes to a physically crosslinked gel. This thermoresponsive gelling property provides an opportunity for formulating an injectable dosage at or below room temperature that can be injected through a needle to a recipient's body and form a firm gel at body temperature as a controlled drug release device.

Several polymeric materials have demonstrated thermoresponsive properties. BASF marketed a series of triblock copolymer of polyethylene oxide (PEO) and polypropylene oxide (PPO) under the trade name of Pluronic™. The polyethylene glycol block is hydrophilic and the polypropylene glycol block is hydrophobic. The Pluronic copolymers are water soluble and the gel shows a reverse thermal gelation property. However, the gel releases drugs in only couple of days and PPO may elicit immunogenic response. Polymers of N-isopropylacrylamide (pNIPA) exhibits a phase transition at about 32° C. in water and can be copolymerized with other polymer to lower LCST. However this polymer is non-biodegradable and not suitable for drug delivery. Yannic B. Schuetz et al., European Journal of Pharmaceutics and Biopharmaceutics 68, 19-25 (2008), reported a chitosan based thermoresponsive hydrogel prepared by treatment of chitosan with acid. The hydrogel made from type I chitosan showed a gelation temperature about 18° C. Although injectable as a fresh solution the reconstituted formulation needs addition of stabilizing agents to maintain thermosetting properties.

Other synthetic block copolymer with amphiphilic characteristics are reported as reverse thermoresponsive polymers. These diblock or triblock copolymers contain PEG as hydrophilic segments and poly-D,L-lactide-co-glycolic acid (PLGA) or poly-L-lactic acid (PLLA) or polypropylene fumarate (PPF) as hydrophobic segments. The representative product is ReGel®, reported by G. M. Zentner et al., Journal of Controlled Release 72, 203-215 (2003), originally disclosed in U.S. Pat. No. 6,004,573, U.S. Pat. No. 7,135,190 B2 and related patents, used in OncoGel™ developed by Macromed (sold to Protherics). ReGel® is an ABA type triblock copolymer having PEG (Mw=1,000 for ReGel'® or 1,450 for ReGel®-2) as the core block B and PLGA as the two side blocks A. A 23% ReGel® aqueous solution is a low viscosity liquid at a low temperature and changes to a high viscosity gel at 13.62° C. ReGel®-2 has similar properties but a higher lower critical solution temperature (LCST) of 42.3° C. ReGel® and ReGel®-2 can be mixed in different ratios to give a mixture with adjusted LCST. However, to get a desirable LCST close to body temperature is difficult.

Churchill J. R. et al., U.S. Pat. No. 4,526,938 and U.S. Pat. No. 4,745,160, described the syntheses and drug release properties in in vitro and in vivo studies of biodegradable AB and ABA block copolymers containing hydrophobic A block and hydrophilic B block. In the examples the hydrophobic A block is PLA or PLGA and the hydrophilic B block is polyethylene glycol (PEG) or polyvinylalcohol (PVA). The described AB type copolymers have PEG blocks with an average molecular weight of 1,900, 5,000 or 5,900 and a PLA/PEG weight ratio of 1:1, 3:1 or 4:1. The AB block copolymer can be dispersed in water (only the copolymer with PEG Mw=1,900) or a mixture of acetic acid and water or water-in-oil dispersions. The illustrated ABA type copolymers contain PEG blocks with an average molecular weight of 2,000, 6,000, or 20,000, with a PLA/PEG or PLGA/PEG weight ratio of 1:1, 3:1, 4:1, 5.7:1, or 19:1. The ABA block copolymers can be dispersed in a mixture of ethanol and water and freeze-dried and pressed into slabs or films as implants or they can be blended with a drug in an aqueous solution. Water solubility of these block copolymers is not demonstrated and organic solvents are used for dissolution, such as acetone and acetic acid. Thermoresponsive behavior is not described and these copolymer-drug mixtures are generally modulated into solid forms at 60° C. as implants for drug release.

B. M. Jeong et al., U.S. Pat. No. 6,841,617 B2, synthesized biodegradable thermoresponsive graft polymers by either reacting two functionalized PEGs followed by a ring opening polymerization using polyester monomers or reacting one functionalized PEG with PLGA monomers. The first procedure is cumbersome which involves three steps in synthesis. The polymers made by the two procedures have weight average molecular weight (Mw) about 11,000 and 9,300 respectively. The in vivo degradation study in rats indicated the presence of the gel at the injection site after 2 months which is relatively long compared with the one month complete degradation of ReGel®.

B. M. Jeong et al. also reported ABA and BAB PEG-PCL block copolymers, Macromolecule, 38, 5260-5265 (2005), and Biomacromolecules, 6, 885-890 (2005), wherein PCL representing poly-ε-caprolactone. The ABA type block copolymer was prepared by polymerization of ε-caprolactone on PEG (Mw=1,000 or 1,500) and the BAB type block polymer by coupling reaction using hexamethylene diisocyanate, a toxic reagent. The ABA block copolymers, such as PII (690-1,000-690) and PIII (980-1,000-980) and a BAB type block polymer (550-2,190-550) showed interesting gelation properties. The degradation properties of these copolymers were not reported in the two articles. However, similar ABA type PCL-PEG-PCL triblock polymers (680-4,000-680) synthesized by L. Martini et al., J. Chem. Soc., Faraday Trans., 90(13), 1961-1966 (1994), showed a very slow in vitro degradation, only about 20% reduction in Mw in 16 weeks.

Su Jeong LEE et al. Journal of Polymer Science: Part A: Polymer Chemistry, 44, 888-899 (2006), and Polymer Journal, 41, 5, 425-431 (2009), reported 4-arm star-shaped PLGA-PEG and PEG-PLGA block copolymers having temperature-sensitive sol-gel transition properties. The PLGA-PEG block copolymer was synthesized by ring-opening polymerization of D,L-lactide and glycolide with glycerol or pentaerythritol as the polyol initiator and coupling of the star-shaped PLGA with carboxyl terminated methoxypolyethylene glycol (MPEG) using N,N′-dicyclohexylcarbodiimide as the coupling reagent. Although these block copolymers show gelation temperatures adequate for thermoresponsive injectable drug formulations, their aqueous solutions require relatively high polymer concentrations to maintain the gelation properties due to their high critical gel concentrations (CGC at 24-34%). The star-shaped 4-arm PEG-PLGA copolymers were synthesized by bulk ring-opening polymerization of D,L-lactide and glycolide in presence of a 4-arm branched PEG as an initiator. The aqueous solutions of the copolymers with lactide and glycolide mole ratio of 3 and degree of polymerization (DP) of 8, in concentrations of 15 wt % and 30 wt % gel at 17 and 11° C. respectively, and lose their gel forms at 23° C. and 31° C., respectively. When the DP was decreased to 7 the sol-to-gel temperature increased about 7-10° C. with little changes in gel-to-sol temperatures. Since the gelling temperatures are low and the upper critical solution temperatures are lower than 37° C. they are not suitable as thermoresponsive depot systems.

The limitations in the currently available themoresponsive polymer systems prompted us to develop a polymer system with the desirable properties and additional improvement in water solubility, drug releasing property, degradation rate, gelation properties such as gel strength and gelation temperature.

### SUMMARY OF THE INVENTION

The present invention refers to a 3-arm PEG-PLGA copolymer comprised of a 3-arm PEG and polyester. The 3-arm PEG is a glycerol ethoxylate (namely ethoxylated glycerol) which is made from glycerol and ethylene oxide. The polyester is polylactide-co-glycolide formed by ring-opening polymerization using the 3-arm PEG as an initiator and D,L-lactide and glycolide as monomers providing the 3-arm PEG-PLGA block copolymer. Other most preferred monomers, which are used to synthesize the products with variable monomer compositions and different physical characteristics include D-lactide, L-lactide, and c-caprolactone. For purpose of easy illustration L (lactide) and G (glycolide) are used in the description of this invention, however, each of which should include all its steric isomers or derivatives of the mentioned monomer. The average molecular weight of the 3-arm PEG in the 3-arm PEG-PLGA is in the range of 1,000 to 4,000 Daltons, and preferably in the range of 1,500 to 3,000 Daltons. The average molecular weight of the 3-arm PEG-PLGA is in the range of 4,000 to 9,000 Daltons, and preferably in the range of 5,000 to 7,500 Daltons. The mole ratio of the monomers and the molecular weight of the 3-arm PEG and polyester length can be fine tuned until a desirable copolymer is identified. In the 3-arm PEG-PLGA copolymer the molar ratio of lactide and glycolide content (L:G ratio) is between about 1:1 and 1:0, preferably between 2:1 and 5:1. The weight ratio of the polyester and PEG is an indicator of the hydrophobicity of the copolymer. A ratio of 2.4 or higher indicates the copolymer is highly hydrophobic and a ratio of 2.1 or lower indicates the copolymer is relatively hydrophilic. The hydrophilicity determines the water solubility of the copolymer and also affects the copolymer's lower critical solution temperature (LOST). Due to the special branched structure the copolymer was found to be able to form a firm gel with lower polyester content about 67% at 30-35° C. Furthermore, the gel of 3-arm copolymer starts to release water gradually until reaching about 50% of its original volume or about 63% of its water content in the temperature range of 40-45° C.

The 3-arm copolymer is soluble in cold water (0-15° C.) up to about 40%. The resulting solutions have low viscosities and are easily filtered as a process in purification and sterilization. The low viscosity at low temperature also facilitates the mixing of polymer solutions with drugs and other additives. At room temperature a formulation with 20% (w/w) of the 3-arm copolymer can be injected through a 25G needle without causing clogging.

As used herein the following terms shall have the assigned meanings. For example, the term “parenteral” shall mean intramuscular, intraperitoneal, intra-abdominal, subcutaneous, and, to the extent feasible, intravenous and intra-arterial.

As used herein, the term “gelation temperature” means the temperature at which an aqueous solution of the biodegradable block copolymer undergoes reverse thermal gelation, i.e. the temperature below which the block copolymer is soluble in water and above which the polymer solution undergoes a phase transition with a rapid increase in viscosity or to form a semi-solid gel.

The terms “gelation temperature” and “reverse thermal gelation temperature” or the like shall be used interchangeably in referring to the gelation temperature.

“Polymer solution,” “aqueous solution” and the like, when used in reference to a biodegradable block copolymer contained in such solution, shall mean a water-based solution having such block copolymer dissolved therein at a functional concentration, and maintained at a temperature below the gelation temperature of the block copolymer.

“Reverse thermal gelation” is the phenomena whereby a solution of a block copolymer spontaneously increases its viscosity, and in many instances transforms into a semi-solid gel, as the temperature of the solution is raised above the gelation temperature of the copolymer. For the purposes of the invention, the term “gel” includes both the semi-solid gel state and the high viscosity state that exists above the gelation temperature. When cooled below the gelation temperature, the gel spontaneously reverses to the low viscosity solution. This cycling between the solution and the gel may be repeated because the sol/gel transition does not involve any change in the chemical composition of the polymer system. All interactions to create the gel are physical in nature and do not involve the formation or breaking of covalent bonds.

“Depot” means a drug delivery system which has changed its form from a liquid to a gel following administration to a warm-blooded animal where the temperature of the liquid is raised to or above the gelation temperature.

“Gel” means the semi-solid phase that spontaneously forms as the temperature of the “polymer solution” or “drug delivery system” is raised to or above its gelation temperature.

“Aqueous composition” or “aqueous drug delivery composition” means either a drug delivery formulation in a liquid or gel form comprised of a water phase containing uniformly therein a drug and the biodegradable block copolymer. At temperatures below the gelation temperature of the block copolymer aqueous solution the copolymer may be soluble in the water phase. At temperatures at or above the gelation temperature the aqueous solution will be solidified to become a gel or semi-solid.

“Aqueous phase” or “water phase” means the continuous water portion in a system which may contain other mixable solvents, solutes and dispersed solids.

“Biodegradable” means that the block copolymer can chemically or enzymatically break down or degrade within the body to form nontoxic components. The rate of degradation can be the same or different from the rate of drug release.

“Mucoadhesive” refers to polymeric materials that adhere to mucosal tissues.

“Drug” shall mean any organic or inorganic compound or biological substance having bioactivity and adapted or used for a therapeutic purpose. Proteins, hormones, anti-cancer agents, oligonucleotides, DNA, RNA and gene therapy agents are included under the broader definition of drug.

“Nutraceuticals” shall refer to a food or food product that provides health and medical benefits including the prevention and treatment of disease. The nutraceuticals include nutrients, dietary supplements and herbal products.

“Ginseng” refers to the plant genus Panax and “ginseng extract” refers to a water or organic solvent extract of the plant parts. Ginsenosides refer to a class of steroid glycosides, and triterpene saponins found exclusively in the root of Panax ginseng C A. MEYER (Araliaceae) and includes the metabolites of ginsenoside-Rg, (Rg,), -Rb, (Rbt) and -Rb₂ (Rb₂).

“Polylactide-co-glycolide” or “PLGA” shall mean a copolymer derived from the condensation copolymerization of lactic acid and glycolic acid, or, by the ring opening polymerization of a-hydroxy acid precursors, such as lactide or glycolide. The terms “lactide” and “lactate,” “glycolide” and “glycolate” are used interchangeably.

“Polylactide” or “PLA” shall mean a polymer derived from the condensation of lactic acid or formed by the ring opening polymerization of lactide.

“Biodegradable polyesters” refers to any biodegradable polyesters, which are preferably synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxyhexonoic acid, γ-butyrolactone, γ-hydroxybutyric acid, δ-valerolactone, δ-hydroxyvaleric acid, hydroxybutyric acids, malic acid, and copolymers thereof.

The pharmaceutical formulation of the present invention may be administered by a variety of methods. Such methods include, by way of example and without limitation: intraperitoneal, intra-articular, intra-arterial, intracardiac, intracavity, intracartilaginous, intradermal, intrathecal, intraocular, intraspinal, intrasynovial, intrathoracic, intratracheal, intrauterine, epidural, percutaneous, intravascular, intravenous, intracoronary, intramuscular or subcutaneous injection; inhalation; or oral, nasal, buccal, rectal, ophthalmic, otic, urethral, vaginal, or sublingual dosage administration. Such methods of administration and others contemplated within the scope of the present invention are known to the skilled artisan.

It is an object of the present invention to provide a biodegradable, 3-arm PEG-PLGA copolymer for use in a drug delivery system which forms a firm gel at body temperature and exhibits controlled drug release properties as a drug release depot.

It is another object of this invention to provide a biodegradable, thermoresponsive, injectable drug delivery system which forms a firm gel at body temperature and exhibits a controllable dehydration property for heat stimulated drug release.

It is another object of this invention to provide a drug delivery system for parental administration of hydrophilic drugs such as peptide, protein and oligonucleotide drugs and hydrophobic drugs such as anticancer drug using water as the solvent or dispersing medium.

It is another object of this invention to provide a method for formulating and administering an aqueous drug solution by injecting or implanting through a needle or catheter into a host body where the liquid formulation gels to form a drug delivery device.

It is another object of this invention to provide a biodegradable copolymer capable of increasing water solubility of a drug, and particularly a hydrophobic drug in a water-based pharmaceutical formulation.

It is another object of this invention to provide a method for administering an aqueous formulation at or below room temperature as an aqueous liquid to an animal with a physiological temperature at about 37° C. where the formulation gels and releases the drug in a controlled manner and for further releasing the drug or another drug by an external heat stimulus via polymer syneresis, that is, heat induced dehydration.

It is another object of this invention to provide a method for anticancer chemotherapy using the invention copolymer as an intratumoral drug-releasing implant after surgical resection of a solid tumor. The implant can be delivered by injecting or spraying an aqueous solution of the invention copolymer and an anticancer drug and formed by thermoresponsive gelling in the surgical cavity.

It is another object of this invention to provide a method for prophylaxis of postoperative infections by using the invention copolymer as an antibiotic releasing implant in surgery. The implant can be delivered by injecting or spraying an aqueous solution of the invention copolymer and an antibiotic and formed by its thermoresponsive gelling in the surgical cavity.

It is another object of this invention to provide a method for the use of an aqueous solution of the invention copolymer as dosage aerosols for therapy of obstructive lung diseases, especially asthma and cystic fibrosis or systemic delivery of insulin. Since chlorofluorocarbons (CFC) used as propellant gases are recognized to be potentially ozone depleting, the use of nebulizers, where aqueous solutions of pharmacologically active substance are sprayed under high pressure of air or oxygen so that a mist of inhalable particles results, is more environment friendly. The advantage of these nebulizers is that they can be completely dispensed without the use of propellant gases such as CFC or hydrofluoroalkanes (HFAs). With the recent advances in nebulizers aqueous drug dispersions or solutions can be aerosolized to form particles with a size of 1-10 μm. Such aqueous aerosols and in particular those of sufficiently small particle size are expected to be inspired into the alveoli of the lung. With the invention polymer aqueous solution the particles deposited on lung tissue will gel and serve as a scattered drug depot system for a sustained drug release. The aerosol form of the invention polymer aqueous solution can also deliver insulin for sustained systemic drug release. For aerosols with droplets of 1-10 μm the drug/polymer will mainly deposit on respiratory tract which can be used to deliver drugs to treat respiratory infections, cystic fibrosis or asthma. To prevent aerosol caused brochoconstriction the aqueous solution needs to be isotonic and with a pH=6.3±0.7.

It is another object of this invention to provide a composition comprising a nutraceutical such as a ginseng product and other ingredients such as a mucoadhesive for prolonging the retention of the composition on mucus and penetration enhancers for improving ginseng absorption. It is another object to provide a method for administration of the above composition with a dosage form of liquid, paste or spray applied to oral mucosa such as sublingual or buccal mucosa to deliver a nutraceutical. Upon contacting mucosa, the composition forms gel and adheres to the mucosa for several hours. This oral transmucosal delivery formulation is particularly valuable for delivering pharmaceuticals and nutraceuticals that are prone to degradation in gastrointestinal tract caused by gastric acid and bacterial enzymes when taken orally. The gelation and adhesion of the composition on the mucosa prolongs the dosage retention and drug/nutrient delivery time of the composition. A drug or nutrient delivered by transmucosal route can be absorbed directly into the systemic circulation bypassing the gastric digestion and hepatic metabolism and thus increasing overall bioavailability.

Additional objects and advantages of this invention will become apparent from the following summary and detailed description of the various embodiments making up this invention.

### BRIEF DESCRIPTION OF THE DRAWINGS

A more complete appreciation of the invention will be readily obtained by reference to the following description and the accompanying drawings, wherein:

FIG. 1 is a phase diagram illustrating the gelation behavior of an aqueous solution of a 3-arm PLGA-PEG copolymer, at different concentrations and temperatures.

FIG. 2 is a release profile of paclitaxel from a 3-arm PLGA-PEG copolymer aqueous gel at 37° C. showing a controlled drug release for 10 days.

FIG. 3 is a release profile of doxorubicin from a 3-arm PLGA-PEG copolymer aqueous gel at 37° C. showing a controlled drug release for 8 days.

### DETAILED DESCRIPTION OF THE INVENTION I

The following descriptions of the invention are provided to illustrate the preferred embodiments, not to limit the invention to a particular polymer composition, molecular weight, block polymer weight ratio, process procedure, monomers, their mole ratio, and reagents. The 3-arm PEG-PLGA was prepared from 3-arm PEG, namely glycerol ethoxylate, which in turn was synthesized from glycerol and ethylene oxide; the synthesis of the 3-arm PEG-PLGA involves ring opening polymerization with the 3-arm PEG as the initiator and D,L-lactide and glycolide as monomers.

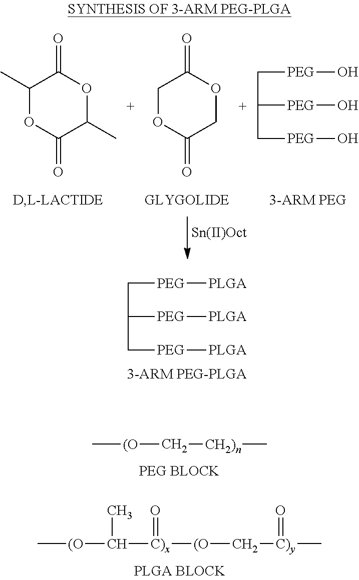

The 3-arm PEG refers to glycerol ethoxylate or ethoxylated glycerol, made from glycerol and ethylene oxide. Glycerol ethoxylate is a commercially available product with different molecular weights. Ethylene oxide is polymerized on the hydroxyl groups of glycerol and 3 polyethylene glycol (PEG) chains form in the glycerol molecule. PLGA refers poly-D,L-lactic acid-co-glycolic acid. Ring-opening polymerization using the 3-arm PEG as an initiator and D,L-lactide and glycolide as monomers provides the 3-arm PEG-PLGA block copolymer. Other most preferred monomers, which are used to synthesize the products with variable monomer compositions and different physical characteristics includes D-lactide, L-lactide, and c-caprolactone. Additional preferred monomers for the synthesis of the copolymers are selected from D,L-lactic acid, D-lactic acid, L-lactic acid, glycolic acid, ε-hydroxy hexonoic acid, γ-butyrolactone, γ-hydroxybutyric acid, δ-valerolactone, δ-hydroxyvaleric acid, hydroxybutyric acids, and malic acid.

These and other objects are achieved by the synthesis of a 3-arm PEG-PLGA copolymer which contains a glycerol derived 3-arm PEG with each PEG arm having a polyester chain at the end. The average molecular weight of the 3-arm PEG in the 3-arm PEG-PLGA is in the range of 1,000 to 4,000 Daltons, and preferably in the range of 1,500 to 3,000 Daltons. The average molecular weight of the 3-arm PEG-PLGA is in the range of 4,000 to 9,000 Daltons, and preferably in the range of 5,000 to 7,500 Daltons. The mole ratio of the monomers and the molecular weight of the 3-arm PEG and polyester length can be fine tuned until a desirable copolymer is identified. In the 3-arm PEG-PLGA copolymer the molar ratio of lactate and glycolate content (L:G ratio) is between about 1:1 and 1:0, preferably between 2:1 and 5:1. Due to the special branched structure the copolymer was found to be able to form a firm gel with lower polyester content (about 67% compared with that of ReGel® at above 70%) at 30-35° C. Furthermore, instead of precipitating as ReGel® does when heated above 40-45° C., the gel of 3-arm copolymer starts to release water gradually while maintains a firm gel form. This observation indicates that the gel of the 3-arm copolymer has strong crosslinking and affinity with water which improve gel strength and stability. In addition, this controllable dehydration property may be useful in heat induced drug release, where an external heat source renders the depot polymer to shrink and releases an embedded drug. Several mechanisms have been utilized to generate heat in depth for treatment of cancerous tumors, such as ultrasound, radiation and magnetic field. A thermosensitive depot system at the site of cancer will respond to a heat stimulus and release water along with a drug. Since the level of heating and duration are controllable, the heat induced drug release would be controllable too. This application may provide a localized and more directly controlled drug release for chemotherapy.

The 3-arm copolymer is soluble in cold water (0-15° C.) up to about 40%. The resulting solutions in concentrations of 10-25% (w/w) have low viscosities and can be easily mixed with a drug and filtered in the fabrication of injectable formulations.

It has been demonstrated that ginseng has a wide range of pharmacological properties including antifatigue and antistress actions, mild normalizing effects on blood pressure and carboyhydrate metabolism, suggesting central nervous system stimulatory properties and effect on macromolecular synthesis in the liver. Further studies suggested that the saponin might be an active principle and ginseng/saponin could stimulate the carbohydrate metabolism in the liver and could increase the lipid content of adipose tissue. It is also believed that the action of ginseng has some special feature in its mode of action and suggested ginseng saponin being a kind of metabolic regulator or hormone-like substance.

### Example 1

### Synthesis of 3-Arm PEG-PLGA

In a glass flask glycerol ethoxylate (Mw=2,000, 1.613 g) was dried under vacuum (1 mmHg) at 150° C. for 3 hours. D,L-lactide (2.829 g) and glycolide (0.568 g) were added to the flask and the mixture was heated with stirring until all solids melt. Polymerization was initiated by the addition of stannous octoate (1 mg). The reaction mixture was heated at 155° C. for 8 hours and cooled to room temperature to give a semi-solid. The interior wall of the flask was rinsed with acetone to remove unreacted monomers and the solid was dried under vacuum. The copolymer was dissolved in cold water to afford a 25% (w/w) solution and separated by heating the solution to 70° C. and decanting the liquid. The purification was repeated twice. After completely dried the copolymer has a weight average molecular weight of 6,820 measured by GPC. GPC was performed on a 4.6×300 mm Styragel HR2 column calibrated with PEG standards using a R1 detector and THF as the eluent.

### Example 2

### Polymer Gelation and Dehydration Properties

The thermoresponsive behavior of aqueous solutions of the 3-arm PEG-PLGA copolymer including gelation properties and heat induced dehydration properties were studied. The copolymer from Example 1 was dissolved in cold water (0-4° C.) by vortexing in a 4 ml glass vial to give 20% (w/w) aqueous solutions, which were placed in a water bath with temperature set at 26° C. The temperature was increased 1° C. each step, in which the viscosity of the solution and transparency of the solution were visually observed. A gelation temperature was recorded if the solution did not flow in 30 seconds upon inverting the sample vial. Dehydration of the gelled solution was monitored by measuring the liquid released from the gel as a cumulative percentage of its original sample volume at different temperatures starting from the gelation temperature up to 50° C. The results of this study were summarized in FIG. 1 and TABLE 1 below.

TABLE 1

| T ° C. | 25 | 33 | 34 | 35 | 37 | 39 | 40 | 42 | 43 | 45 | 46 | 47 |
|---|---|---|---|---|---|---|---|---|---|---|---|---|
| Transparency | C | O | O | O | O | O | O | O | O | O | O | O |
| form | L | G | G | G | G | G | G | G | G | G | G | G |
| liquid | 0 | 0 | 0 | 0 | 0 | 0 | 10 | 20 | 30 | 50 | 53 | 55 |
| released % | | | | | | | | | | | | |

Footnote: C: clear, O: opaque, L: liquid, G: gel.

### Example 3

### Paclitaxel Release

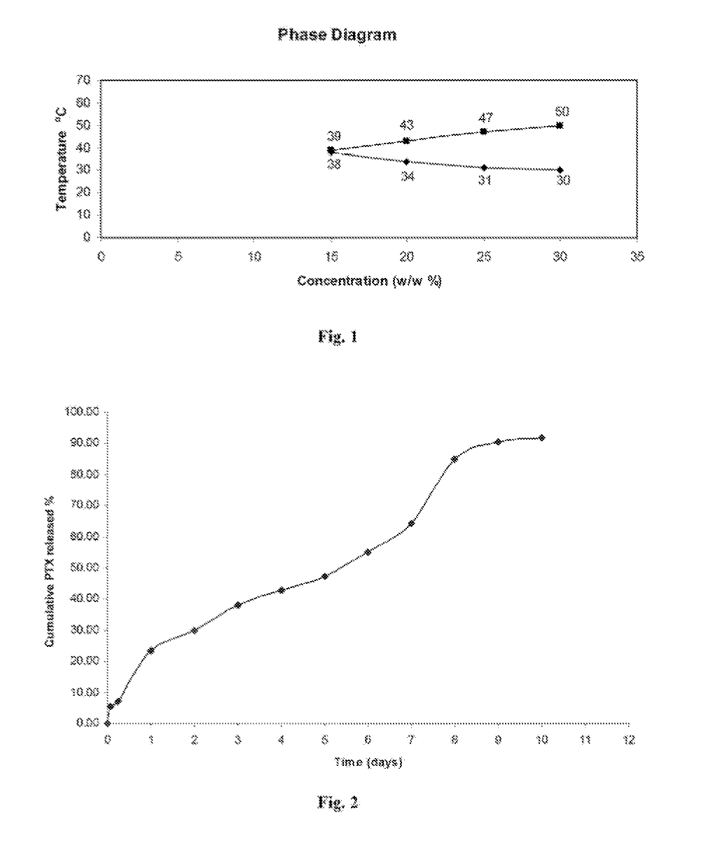

Paclitaxel was used as a hydrophobic drug in a drug release study using the copolymer of EXAMPLE 1 in a 20% (w/w) aqueous solution. The drug (4 mg) was added to a 4 ml vial containing 0.2 ml of the polymer solution. At 4° C. the sample vial was sonicated for 15 min and vortexed for 16 h and sonicated for 15 min again to give a slightly cloudy suspension. After filtered through a 0.2 μm syringe filter an aliquot (0.2 ml) of the solution was added to each sample vial. The triplet samples were placed in a water bath preheated and stabilized at 37° C. After 10 min a buffer solution of 1×PBS (3 ml, preheated at 37° C.) containing 2.4% (w/w) of Tween 80 and 4% (w/w) of Cremophor EL was added to each sample vial containing a gelled drug/polymer solution. The drug release was determined by taking an aliquot of the medium from each sample vial at 1.5 h, 6 h and then daily for 10 days and analyzing the drug content of the sample by HPLC (mobile phase 1 ml/min 50/50 acetonitrile/deioned water, wavelength 227 nm, running time 15 min). The drug release profile is shown in FIG. 2.

### Example 3

### Doxorubicin Release

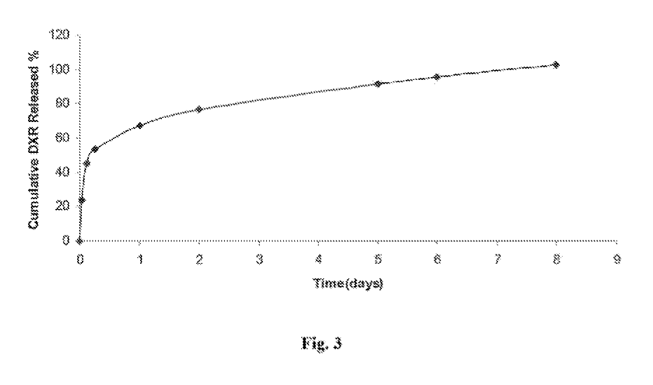

Doxorubicin was used as a hydrophilic drug in a drug release study using the copolymer of EXAMPLE 1 in a 20% (w/w) aqueous solution. The drug (4 mg) was dissolved in 2 ml of the polymer solution. The drug/polymer solution was filtered through a 0.2 μm syringe and an aliquot of 0.5 ml of the solution was added to each sample vial. The triplet samples were placed in a water bath preheated and stabilized at 37° C. After 10 min a buffer solution of 1×PBS (1 ml, preheated at 37° C.) was added to each sample vial containing a gelled drug/polymer solution. The drug contents in the release media, which were removed and replaced with fresh PBS solutions at each sampling point, were determined at 1, 3, 6 h and then daily for 8 days by a spectrophotometer (SpecraMax M2 by Molecular Devices). The drug release profile is shown in FIG. 3.

### Example 4

### Enhanced Drug Solubility in Polymer Gel

In a 4 ml vial the polymer from EXAMPLE 1 (200 mg) and paclitaxel (2 mg) were dissolved in acetone (1·ml). The mixture was evaporated under vacuum for 16 h. Deionized water (0.8 ml) was added and the mixture was stirred at 4° C. for 24 h. The resulting solution was filtered through a 0.2 μm syringe filter to give a clear solution. An aliquot of the solution was diluted 100-fold with acetonitrile and analyzed for paclitaxel by HPLC (mobile phase: water/acetonitrile 1:1, 1 ml/min, injection volume: 20 μl, running time: 15 min, column: Waters Symmetry 300™ 4.6×250 mm, C18 5 μm, detector: Waters 2996). Result: the paclitaxel content in the aqueous solution (20% w/w) was 6.4 mg/ml.

### Example 5

### Polymer Gel Viscosity

The viscosity of a 20% (w/w) gel solution of the polymer from EXAMPLE 1 was measured using Brookfield Viscometer LVDVE, S21 spindle, SC4-13R chamber, circulating water bath and water jacket at 25° C. Result: 20cP at 60 rpm.

The present invention is a novel therapeutic compound that combines the biodegradable thermoresponsive 3-arm polyethylene glycol poly(lactide-co-glycolide) copolymer with the pharmacokinetic ginseng or ginseng saponins and ginsenosides for human absorption and distribution. The media for which the novel therapeutic compound can be distributed includes, but is not limited to, dissolvable membranes for oral administration, sprays for nasal or oral absorption, and chewing gums for oral administration.

We claim:

1. An aqueous biodegradable polymeric ginseng delivery composition capable of gelling at the body temperature of a warm-blooded animal comprising:
(a) a ginseng product in an biologically effective amount; and
(b) a biodegradable 3-arm block copolymer of the following formula: 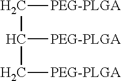
wherein PEG is polyethylene glycol and PLGA is poly(lactide-co-glycolide), and said 3-arm block copolymer and said ginseng product are uniformly contained in the aqueous phase forming a homogeneous liquid, said 3-arm polyethylene glycol has a weight average molecular weight of between about 1,000 and 4,000 Daltons, and said polyester comprises about 20-100 mole percentage of lactate and about 0-80 mole percentage of glycolate, and said biodegradable 3-arm block copolymer has a weight average molecular weight of between about 4,000 and 9,000 Daltons, and the weight ratio of said polyester and said 3-arm polyethylene glycol is between about 1.5 and 3, and said block copolymer content in said aqueous composition is between about 5 and 50% (w/w).

2. An aqueous drug delivery composition according to claim 1 wherein said ginseng product consist of a ginseng extract or a mixture of ginsenosides or a purified ginsenodide.

3. An aqueous drug delivery composition according to claim 1 wherein said composition further comprises one or more additives selected from mucoadhesive, mucosal penetration enhancers, stabilizers, flavors, other nutrients and preservatives.

4. A method for the transmucosal administration and delivery of a ginseng product to a human being comprising:
a) preparing an aqueous composition comprising:
b) a ginseng product in an biologically effective amount;
c) a biodegradable 3-arm block copolymer of the following formula; 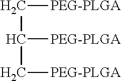
wherein PEG is polyethylene glycol and PLGA is poly(lactide-co-glycolide) and said 3-arm block copolymer and said ginseng product are uniformly contained in the aqueous phase forming a homogeneous liquid, said 3-arm polyethylene glycol has a weight average molecular weight of between about 1,000 and 4,000 Daltons and said biodegradable 3-arm block copolymer has a weight average molecular weight of between about 4,000 and 9,000 Daltons, and said polyester comprises about 20-100 mole percentage of lactate and about 0-80 mole percentage of glycolate, and the weight ratio of said polyester and said 3-arm polyethylene glycol is between about 1.5 and 3;
d) maintaining the temperature of said aqueous composition below its gelation temperature, wherein said block copolymer content of said aqueous composition is between about 5 and 50% (w/w); and
e) administering said aqueous composition as a liquid at a temperature below its gelation temperature to an oral cavity mucosal tissue including buccal, sublingual, and esophageal tissues, by spraying or dripping of said liquid composition, whereon the liquid gels and adheres to the application site for sustained ginseng delivery.

5. A method for the transmucosal administration and delivery of a ginseng product according to claim 4, wherein administration and delivery is accomplished by using a chewing gum media for oral administration.

6. A method for the transmucosal administration and delivery of a ginseng product according to claim 4, wherein administration and delivery is accomplished by using by using a spray media for nasal or oral administration.

7. A method for the transmucosal administration and delivery of a ginseng product according to claim 4, wherein administration and delivery are accomplished by using by using a dissolvable membrane media for oral administration.
